(19) United States
(12) Patent Application Publication
(10) Pub. No.: US 2009/0123638 A1
(43) Pub. Date: May 14, 2009

(54) Monosaccharide production system

(76) Inventor: Aharon Meir Eyal, Jerusalem (IL)

Correspondence Address: CARGILL, INCORPORATED, LAW/24, 15407 MCGINTY ROAD WEST, WAYZATA, MN, 55391, US

(21) Appl. No.: 11/991,509

(22) PCT Filed: Nov. 22, 2005
(86) PCT No.: PCT/US05/42497

§ 371 Date: Jan. 27, 2009

Related U.S. Application Data
(60) Provisional application No. 60/630,137, filed on Nov. 22, 2004.

(51) Int. Cl. A23L 1/48; C07H 1/08; C12P 19/02; C12P 7/06; C12P 7/40; C12P 13/04; C12P 21/00; C07H 3/02; C07H 1/06

(52) U.S. Cl. 426/658; 536/128; 435/105; 435/161; 435/136; 435/129; 435/71.1; 536/125; 536/1.11

(57) ABSTRACT

A monosaccharide production system is disclosed. The production system can be directed to processes for producing a D-galactose preparation, a D-galactose preparation and an isoflavones preparation, a tagatose preparation, and a tagatose preparation and an isoflavones preparation.

### CROSS REFERENCE TO RELATED APPLICATIONS

The following application is cross-referenced and hereby incorporated by reference in its entirety: U.S. patent application Ser. No. 10/820,757, filed Apr. 9, 2004. This application claims the benefit of U.S. Provisional Application No. 60/630,137 filed Nov. 22, 2004, which is incorporated herein by reference in its entirety.

### FIELD OF THE INVENTION

The present invention generally relates to a monosaccharide production system. The present invention more particularly relates to a system and method for producing D-galactose and/or tagatose.

### BACKGROUND OF THE INVENTION

D-galactose is widely used as a raw material in certain industries. For example, many sweeteners (such as polyol sugars) use D-galactose as a raw material for manufacture thereof. In addition, D-galactose can be used in the beverage industry (e.g. in sport drinks as replacement of phenols in resins), in the manufacture of contrast agents, as sweetener in foods (e.g. to prevent tooth decay), etc.

Several methods of preparing D-galactose are known. One conventional method of preparing D-galactose includes the hydrolysis of milk sugar (i.e. lactose). However, such conventional method has several disadvantages including: (a) the milk supply is limited; (b) the milk supply is prone to contamination (e.g. microbial, viral, antibiotic, heavy metal, etc.); and (c) lactose may not be suitable as an ingredient in foodstuffs for people suffering from milk intolerance or in foodstuffs prepared as kosher food.

A conventional method of providing D-galactose in feed includes hydrolyzing galactooligosaccharides from soya bean and canola meal. However, such conventional method is conducted in vivo (i.e. in the gastrointestinal tract of poultry) and does not provide a commercial source of monosaccharide D-galactose preparation.

Accordingly, there is a need for a monosaccharide production system that uses widely available starting material. There is also a need for a monosaccharide production system that yields relatively large and/or pure amounts of D-galactose in monosaccharide form. There is also a need for a monosaccharide producing system that yields relatively large and/or pure amounts of tagatose in monosaccharide form. It would be advantageous to provide a monosaccharide production system filling any one or more of these needs or having other advantageous features.

### SUMMARY OF THE INVENTION

The present invention is directed to processes for producing monosaccharides. In one embodiment is described a process for producing a D-galactose preparation that includes the steps of providing a legume material comprising a D-galactose comprising oligosaccharide, separating oligosaccharide from the legume material to provide an oligosaccharide composition, wherein the oligosaccharide composition comprises at least about 20 percent of the D-galactose comprising oligosaccharide relative to the legume material, treating the oligosaccharide composition to purify the oligosaccharide, and hydrolyzing oligosaccharide to provide a D-galactose preparation. In another embodiment is described a process for producing a D-galactose preparation and an isoflavones preparation that includes the steps of providing an extract of defatted soy flakes, hydrolyzing at least a fraction of D-galactose-containing oligosaccharide from the extract, separating the extract into at least two streams, one of which is enriched in D-galactose compared with isoflavones and the other is enriched in isoflavones compared with D-galactose, purifying the D-galactose-enriched stream, and purifying the isoflavones-enriched stream.

Alternatively, the present invention describes a process for the production of a tagatose preparation that includes the steps of providing a legume extract comprising at least one D-galactose-comprising oligosaccharide, hydrolyzing at least a fraction of the D-galactose-comprising oligosaccharide to form a D-galactose-comprising aqueous solution, isomerizing at least a fraction of the D-galactose in the aqueous solution to form a tagatose-comprising aqueous solution, and purifying the tagatose in the solution. In still another embodiment is a process for producing a tagatose preparation and an isoflavones preparation that includes the steps of providing an extract of defatted soy flakes, hydrolyzing at least a fraction of D-galactose-containing oligosaccharide from the extract, isomerizing D-galactose to tagatose, separating the extract into at least two streams, one of which is enriched in D-galactose or in tagatose compared with isoflavones and the other is enriched in isoflavones compared with D-galactose or tagatose, purifying the D-galactose or tagatose-enriched stream, and purifying the isoflavones-enriched stream.

### BRIEF DESCRIPTION OF THE DRAWINGS

FIG. 1 is a flow diagram of a monosaccharide isolation system according to an exemplary embodiment of the present invention.

### DETAILED DESCRIPTION

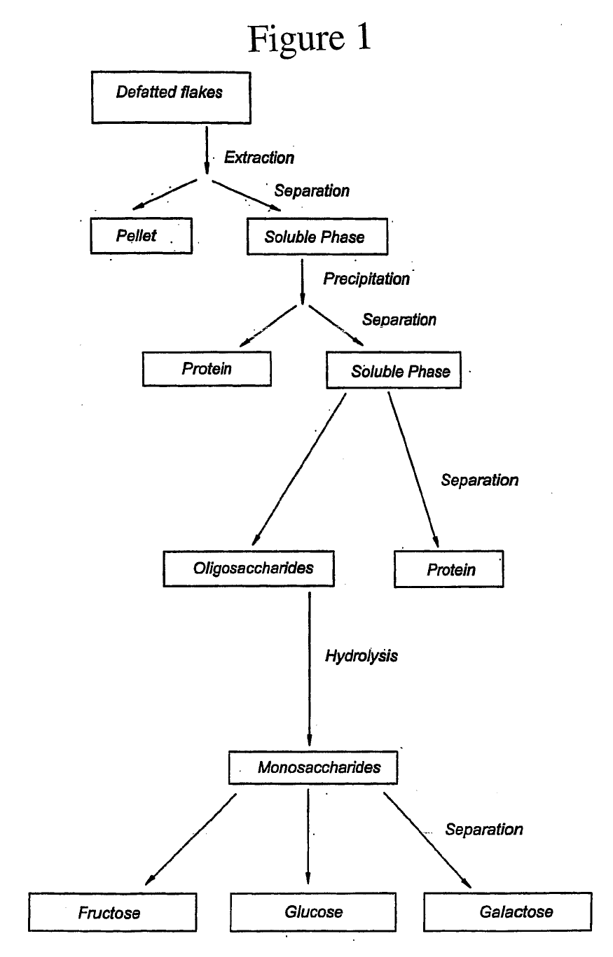

A monosaccharide producing system is shown in FIG. 1 according to an exemplary embodiment. The system includes a method of producing D-galactose from legume material. Referring to FIG. 1, the method includes subjecting a legume composition having a galactose-containing oligosaccharide to one or more treatments, resulting in a preparation having oligosaccharides (an oligosaccharide composition), and changing or converting the oligosaccharides to monosaccharides by hydrolysis.

As used in this disclosure, the term “galactose-containing (or galactose-comprising) oligosaccharide” means and includes an oligosaccharide having a galactose moiety and a moiety of a different monosaccharide (e.g. glucose, fructose, etc.). The term “galactose-containing (or galactose-comprising) oligosaccharide” also means and includes non-homologous galactose polymers.

According to a preferred embodiment, at least one of the oligosaccharides in the oligosaccharide composition is a galactose-containing oligosaccharide. According to a suitable embodiment, at least about 50 percent of the galactose-containing oligosaccharides have no other monosaccharide moiety besides fructose and glucose, suitably at least about 75 percent, more suitably at least about 90 percent, more suitably at least about 95 percent, more suitably at least about 99 percent. According to a suitable embodiment, at least about 30 percent by weight of the oligosaccharides in the oligosaccharide composition are galactose-containing oligosaccharides, suitably at least about 40 percent by weight, more suitably at least about 50 percent by weight. According to a suitable embodiment, the oligosaccharide composition comprises at least about 20 percent of the D-galactose-comprising oligosaccharides of the legume material, suitably at least about 40 percent, more suitably at least about 60 percent. According to a suitable embodiment, the treated oligosaccharide composition comprises at least about 20 percent D-galactose-comprising oligosaccharides on a dry weight basis, suitably at least about 40 percent, more suitably at least about 50 percent. According to another suitable embodiment, at least about 60 percent of the galactose-containing oligosaccharides are hydrolyzed to D-galactose in monosaccharide form, suitably at least about 70 percent, more suitably at least about 80 percent.

The method of isolating D-galactose does not use non-homologous galactose polymers as a source for D-galactose according to a preferred embodiment—rather the method uses non-homologous sugar polymers or oligomer as a source for D-galactose. According to a preferred embodiment, rare sugars are absent from the oligosaccharides (e.g. those sugars that are not typically used in foodstuffs such as arabinose, rhamnose, fucose, mannose, galactose variants other than D-galactose, type I arabinogalactan, type II arabinogalactan, uronic acids, etc.). The method produces a compound or preparation having D-galactose in monosaccharide form that can be readily used in the food industry or fermentation industry according to a preferred embodiment. The compound having D-galactose in monosaccharide form can be subjected to additional purification steps, generating a composition with an elevated content of D-galactose, applicable in for example the food, cosmetic, fermentation industry, chemical industry, etc. according to alternative embodiments.

The compounds having D-galactose in monosaccharide form can have, for example: (a) D-galactose and other components being mostly protein, D-glucose and/or D-fructose; (b) D-galactose and other components being mostly D-fructose and/or D-glucose; and/or (c) an increased content of D-galactose, depending on the purity desired for the industrial utilization.

Any legume plant (or plant part) having galactose-containing oligosaccharides may be used as a source material of the D-galactose. Such plants include oilseed vegetables and plants producing beans or peas. Some examples of legume sources having oligosaccharides include sunflower, rape, lupin, soybean cowpeas, etc.

The source used as the starting material for the oligosaccharides may be derived from two or more plants (and/or plant parts). Furthermore, the source of the oligosaccharides may be derived from the original plant (or plant part) via treatments such as dehulling or removal of husk or such like, flaking, the removal of at least part of the fat or oil content, and/or milling, grinding, etc. Furthermore, treatment might include the removal of at least part of the protein, fibers, or starch present. The material may be in any suitable form (e.g. grits, flakes, flour or meal, etc.).

According to exemplary embodiments, the oligosaccharides are extracted from the vegetable source using aqueous extractant, with or without water-soluble organic solvents and/or with or without dissolved salts. Suitable extracts include soy solubles, soy molasses and soy whey. According to a preferred embodiment, the extract is soy molasses obtained on preparing soy protein isolate by membrane filtration of proteins extracted from flakes of defatted soy. The extract having the oligosaccharides may contain a high content of protein, fiber, or starch when compared to the oligosaccharides. When the extract has high protein, fiber or starch contents, additional treatment before hydrolysis of the oligosaccharides (e.g. in the form of removal of protein, fiber or starch) may be desired.

Treatment for the removal of these components can be any suitable treatment such as extraction, centrifugation, decanting, membrane filtration, etc. according to any preferred or alternative embodiments. Such treatments may be carried out singly or in combination. Such treatments may also be further combined with other techniques such as isoelectric protein precipitation.

According to a preferred embodiment, defatted legume material is mixed with water to dissolve the oligosaccharides. From this mixture, the insoluble phase is removed from the soluble phase (e.g. by decanting). The soluble phase may then be treated to precipitate the proteins (e.g. using acid), while the oligosaccharides remain solvated. The insolubilized proteins are then removed (e.g. by centrifugation). Alternatively, soluble protein and other high molecular weight compounds are separated using membrane filtration such as ultrafiltration. Suitable membranes are typically of molecular weight cut-off greater than about 5000 Daltons. The proteins and high molecular weight components are retained on the membranes while the oligosaccharides transfer with the permeate.

Solutions containing oligosaccharides, e.g. ones formed by extraction from legume material such as soy, also contain mono- and disaccharides. Typically, those include fructose, glucose and sucrose. According to a preferred embodiment, those are separated or removed from the oligosaccharides prior to hydrolysis in order to form a purified oligosaccharide composition. According to an exemplary embodiment, the oligosaccharide composition is an aqueous solution and oligosaccharides are crystallized out of it leaving impurities, such as mono- and disaccharides, in the solution. Crystallized oligosaccharides can be separated from the impurities-containing solution, e.g. by decantation or centrifugation and then, if desired, re-dissolved for further treatment. According to a preferred embodiment, oligosaccharide crystallization is solvent aided using for example the addition of a water-soluble solvent such as ethanol to lower the water activity.

According to another exemplary embodiment, the oligosaccharide composition is an aqueous solution and oligosaccharides are separated from mono- and disaccharides and other low molecular weight impurities, such as minerals, by nano-filtration. Using nano-filtration membranes with molecular weight cut-off of about 500 Daltons excludes the oligosaccharides in the retentate, while impurities transfer into the permeate and are separated thereby.

According to still another exemplary embodiment, mono- and disaccharides and other carbon compounds are removed from the oligosaccharide composition by fermentation. The composition may be contacted with organisms capable of metabolizing compounds such as sucrose, glucose and fructose. Preferably, fermentation products are formed. Preferred fermentation products include ethanol, carboxylic acid, amino acids, single-cell protein and enzymes. Optionally, those fermentation products are separated from the oligosaccharides prior to hydrolysis, e.g. by distillation of volatile products such as ethanol, crystallization, adsorption, chromatography, decantation or centrifugation of non-soluble products such as biomass, membrane filtration, e.g. in the case of enzymes production, etc. Alternatively, fermentation products are left with the oligosaccharides and provided together to the hydrolysis step to be separated after hydrolysis or left in the product preparation. According to a preferred embodiment, the fermentation product is an enzyme suitable for hydrolysis of oligosaccharides. Optionally, such enzyme may be separated from the fermentation medium for use in hydrolysis. Alternatively it may be provided to the hydrolysis step with the oligosaccharides. The preparation thus obtained comprises at least about 30 percent of the oligosaccharides on dry weight basis according to a particularly preferred embodiment.

The oligosaccharides in the treated oligosaccharide composition, typically in a soluble phase, are then subsequently hydrolyzed. The hydrolysis may be catalyzed chemically or enzymatically. An example for chemical catalysis is conducting the hydrolysis in an acid solution hydrolysis releases monosaccharides from the specific oligosaccharides present in the preparation.

According to an alternative embodiment, enzymes having the ability to break both alpha-galactosidic linkage and the ability to break beta-fructofuranosidic linkage (and/or a mixture of enzymes comprising the ability to break alpha-galactosidic linkage and the ability to break beta-fructofuranosidic linkage) may be added to the extracted fraction as a hydrolyzing agent. According to another alternative embodiment, the hydrolysis may be accomplished by holding the mixture having the enzymatic hydrolyzing agent and the oligosaccharides at a holding temperature between about 100 C and 900 C for about 5 to 250 minutes, more suitably at a holding temperature between about 200 C and 600 C for about 10 to 100 minutes.

According to suitable embodiments, the oligosaccharides present in the soluble fraction are further purified before a hydrolyzing agent is added. Further purification of the oligosaccharides before hydrolysis may be accomplished using any convenient separation technique, for example membrane separation techniques (e.g. ultrafiltration, diafiltration, microfiltration, nanofiltration, hyperfiltration, etc.), chromatographic techniques, and/or a combination thereof. When using ultrafiltration, suitable membranes may have a theoretical molecular weight cut-off of about 1,000 to about 200,000 Daltons, suitably from about 2,000 to about 50,000 Daltons, more suitably from about 5,000 to 35,000 Daltons.

According to a preferred embodiment, the product of hydrolysis is further purified in order to increase D-galactose concentration there. Increase in the purity of the resulting D-galactose in monosaccharide form may be accomplished by separating D-galactose from other saccharides or by separating D-galactose from non-saccharides or combinations thereof. According to a suitable embodiment, first the (residual) non-saccharides are separated from the D-galactose. The resulting saccharide mix comprises mainly monosaccharides and can be used in various types of industry. Subsequently, the content of D-galactose can be further increased by removal of the other saccharides. The rest or remainder streams of this process will thus be enriched in other monosaccharides such as D-fructose and D-glucose according to a preferred embodiment. D-galactose can, for example, be separated from such other saccharides using techniques such as crystallization, e.g. solvent-aided crystallization and chromatography. Additionally, chromatography may also be used for increasing the concentration of D-galactose in the preparation by removal of salt, protein, or fibrous components using known technology. In addition, techniques using active charcoal and crystallization may be used to increase the D-galactose content. A particularly preferred method for increasing the concentration of D-galactose is fermenting mono-saccharides, such as glucose and fructose and of disaccharides, mainly sucrose, by microorganisms metabolizing these sugars preferably over D-galactose. As in the case of using fermentation for increasing oligosaccharides concentration in oligosaccharides composition, fermentation preferably produces a commercial fermentation product. The preferred fermentation products, e.g. ethanol, carboxylic acids, amino acids, enzymes and single-cell proteins and methods of their separation, in case separation is desired, are similar.

In the hydrolysis product, the monosacharides are suitably present as at least about 60 percent of the total monosaccharide content derivable in theory from the oligosaccharides, more suitably in more than about 70 percent, more suitably more than about 80 percent. According to a suitable embodiment, the concentration of D-galactose in a solution formed by hydrolysis and purification is at least about 10 percent on dry basis, preferably more than about 40 percent, most preferably more than about 90 percent.

According to a preferred embodiment, the production of D-galactose from extracts of legume material, particularly from molasses of producing purified soy proteins, is integrated with the separation of isoflavones from the same sources. Optionally, the extract is treated to separate components other than isoflavones and oligosaccharides by methods such as described above. The treated extract may be then separated into at least two streams, one of which is enriched in isoflavones and the other is enriched with oligosaccharides. In the isoflavones-enriched stream, the ratio between isoflavones and oligosaccharides is greater than that ratio in the treated extract, while in the oligosaccharides-enriched stream that ratio is smaller than that in the treated extract. Alternatively, oligosaccharides in the treated extract may be first hydrolyzed by methods similar to ones described above. The hydrolysis product or a solution derived from it is then separated into at least two streams, one of which is enriched in isoflavones and the other is enriched in D-galactose. In the isoflavones-enriched stream, the ratio between isoflavones and D-galactose is greater than that ratio in the hydrolysis product, while in the D-galactose-enriched stream that ratio is smaller than that in the hydrolysis product. Optionally, additional purification steps may be introduced, e.g. prior to separation into at least two streams, prior to hydrolysis or after it. Purification steps may be conducted also to further purify at least one of isoflavones-enriched stream, oligosaccharides-enriched stream and D-galactose enriched stream according to alternative embodiments.

According to a preferred embodiment, D-galactose in preparations are further processed to form derivates. Such further processing may be conducted after purification of the D-galactose-containing stream or prior to it. In the latter case, purification may be conducted on the product of processing, which in some cases is easier to purify than D-galactose. Suitable separation methods include crystallization, chromatography and fermentation of glucose, fructose and sucrose. According to a preferred embodiment, such further processing involves hydrogenation to form the corresponding sugar alcohol. D-galactose may also be oxidized, esterified with carboxylic or fatty acids and etherified with short- or long-chain alkanols.

According to a preferred embodiment, D-galactose is isomerized to tagatose, which can be used as sweetening, a bulking agent, as probiotic food component, anti-hyperglycemic agent, enhancer of blood factors, synergiser and flavor enhancer. Tagatose can also be used in mixtures with other sugars, e.g. with glucose, fructose or both. Tagatose can also form a suitable platform molecule for other chiral products of commercial application, e.g. sorbose, talitol and 1-deoxygalactonojirimycin. Tagatose is a low-calorie, full-bulk natural sugar, and has attained GRAS (Generally Recognized As Safe) status under U.S. Food and Drug Administration (FDA) regulations, thereby permitting its use as a sweetener in foods and beverages. Tagatose has food and beverage applications and potential health and medical benefits. Various applications of tagatose include use as a low-calorie, full-bulk sweetener in a wide variety of foods, beverages, health foods, and dietary supplements. Tagatose may be used as a low-calorie sweetener in products in which the bulk of sugar is important, such as chocolates, chewing gum, cakes, ice cream, and frosted cereals. The synergism of tagatose with high-intensity sweeteners also makes it useful in sodas. Various health and medical benefits of tagatose may include the treatment of type 2 diabetes, hyperglycemia, anemia, and hemophilia and the improvement of fetal development.

According to an embodiment, tagatose production involves the steps of extracting D-galactose comprising oligosaccharides from legume material, hydrolyzing oligosaccharides to form a D-galactose-containing preparation and isomerizing the D-galactose in such preparation to form a tagatose-containing preparation.

Tagatose is a 6-carbon sugar in the keto form, i.e. C1 (the first carbon atom) carries an hydroxyl and C2 is a carbonyl (C═O) with no hydrogen atom bound to it: CH2(OH)C(═O)Rt, where Rt denotes the rest of the tagatose molecule. Tagatose relates to other sugars in various ways (e.g. being a stereo-isomer of fructose on C4), but the most relevant comparison for the purpose of the present invention is that to galactose. The two sugars differ on the first two carbons, but are identical on the other four, since galactose is C(═O)HCH(OH)Rt. The two sugars related to each other in the same way as fructose relates to glucose. Thus, tagatose is obtainable from galactose by reduction of C1 and oxidation of C2, i.e. rearrangement of the molecule with no consumption of oxygen or other reagents.

Galactose may be converted or isomerized to tagatose using chemical catalysis or bio-catalysis. Chemical catalysis can use salts, preferably CaCl2, preferably at a temperature in the range of between about 0 C and 100 C and preferably in basic conditions. Biocatalyzed conversion can use L-arabinose isomerase. The enzyme may be used as such, immobilized on a support, such as agarose, or in the form of a whole-cell fermentation. A metal ion activator may be used with the enzyme.

Purification and enrichment steps, as described above, are preferably added to form a purified tagatose preparation. Purification steps may be used to concentrate oligosaccharides in the extract, to concentrate D-galactose in the hydrolysis product and/or to purify tagatose in the isomerization product. Purification process may also be used for shifting the equilibrium in the hydrolysis and/or the isomerization step, therefore preferably conducted simultaneously with those conversions. Suitable purification methods include crystallization of oligosaccharides, of sucrose or of monosaccharides. Solvent-aided crystallization is the preferred embodiment in some of the cases. According to another preferred method, tagatose forms a low-solubility complex with Ca(OH)2 which is precipitated, separated and then acidulated to form an insoluble calcium salt and a solution of tagatose. Alternative or additional technologies include ultrafiltration, nano-filtration, ion-exchange, adsorption, treatment with a de-colorant, chromatography and fermentation of sucrose, glucose and/or fructose.

According to a preferred embodiment, the production of tagatose from extracts of legume material, particularly from molasses of producing purified soy proteins, is integrated with the separation of isoflavones from the same sources. Optionally, the extract is treated to separate components other than isoflavones and oligosaccharides by methods such as those described above. The treated extract is then separated into at least two streams, one of which is enriched in isoflavones and the other is enriched with oligosaccharides. In the isoflavones-enriched stream, the ratio between isoflavones and oligosaccharides is greater than that ratio in the treated extract, while in the oligosaccharides-enriched stream that ratio is smaller than that in the treated extract. Oligosaccharides in the oligosaccharides-enriched stream are then hydrolyzed to form D-galactose, which is then isomerized to tagatose. Alternatively, oligosaccharides in the treated extract are first hydrolyzed by methods similar to ones described above. The hydrolysis product or a solution derived from it is then separated into at least two streams, one of which is enriched in isoflavones and the other is enriched in D-galactose. In the isoflavones-enriched stream, the ratio between isoflavones and D-galactose is greater than that ratio in the hydrolysis product, while in the D-galactose-enriched stream that ratio is smaller than that in the hydrolysis product. D-galactose in the D-galactose-enriched stream is then isomerized to tagatose. According to still another alternative embodiment, oligosaccharides in the treated extract are first hydrolyzed by methods similar to ones described above. D-galactose in the hydrolysis product is isomerized to tagatose. The isomerization product is then separated into at least two streams, one of which is enriched in isoflavones and the other is enriched in tagatose. In the isoflavones-enriched stream, the ratio between isoflavones and tagatose is greater than that ratio in the hydrolysis product, while in the tagatose-enriched stream that ratio is smaller than that in the hydrolysis product. Optionally, additional purification steps are introduce, e.g. prior to separation into at least two streams, prior to hydrolysis or after it. Purification steps may be conducted also to further purify at least one of isoflavones-enriched stream, oligosaccharides-enriched stream, D-galactose enriched stream and tagatose-enriched stream.

### EXAMPLES

While the invention will now be described in connection with certain embodiments in the following examples so that aspects thereof may be more fully understood and appreciated, the examples are not intended to limit the invention to these particular examples.

### Example 1

The process may be carried out at a commercially attractive scale, i.e. at least on a scale equal to pilot scale. On a pilot plant scale, 150 liters of water having a temperature of about 200 C may be added to 30 kg defatted soybean flakes and stirred to form a uniform mixture. After 20 minutes, insoluble components in the mixture may be separated from the soluble fraction by decanting. Hydrochloric acid may be added to the soluble fraction to conduct acid precipitation at pH 4.5, followed by centrifugation at 7800×g to separate the insolubles from the solubles. The soluble fraction may be further treated using ultrafiltration. The ultrafiltration membrane may have a theoretical molecular weight cut-off of 5.000 Daltons. The retentate may be separated from the permeate, which may contain soluble saccharides. The permeate is expected to contain about 50% saccharides on dry weight basis.

0.1% Alpha-gal 600L (Novo-Nordisk) based on dry matter may be added. (Alpha-gal 600L is an enzyme preparation containing both alpha-galactosidic activity and betafructofuranosidic activity). The polysaccharides within the permeate soluble saccharides on dry weight basis may be hydrolyzed to a preparation containing mainly monosaccharides, by incubating the mixture for 4 hours at 500 C. The D-galactose content of the preparation thus obtained is expected to be about 5-10% on dry weight basis.

### Example 2

Defatted soybean flakes are treated with 10 times their weight of water to which NaOH may be added to adjust the pH to 8.5. After an hour of mixing, the solution is separated from insolubles by centrifugation. The separated solution is treated with an ultrafiltration membrane with molecular weight cut-off of 30,000 Daltons. Proteins and other high molecular weight solutes are retained on the membranes, while the oligosaccharides permeate through the membrane. The permeate also contains sucrose, fructose and glucose. The permeate is treated with a yeast fermenting those sugars to ethanol. Most of the ethanol is distilled out of the yeast-treated permeate. The aqueous solution after the distillation of ethanol is treated with Alpha-gal as in Example 1 to hydrolyze the oligosaccharides contained in it. The D-galactose content of the preparation obtained is expected to be about 15-25 percent.

### Example 3

CaCl2 and Ca(OH)2 are added to D-galactose preparation formed according to Example 2 and the temperature is adjusted to 25 C. Galactose is converted to tagatose, which forms a complex with Ca(OH)2.

### Example 4

L-arabinose isomerase supported on agarose is added to D-galactose preparation formed according to Example 2. About 30 percent of the galactose contained in the solution is expected to be converted to tagatose.

While the preferred and other exemplary embodiments described in this disclosure are presently preferred, it should be understood that these embodiments are offered by way of example only. The invention is not limited to a particular embodiment, but extends to various modifications, combinations, and permutations.

What is claimed is:

1. A process for producing a D-galactose preparation comprising:
a. providing a legume material comprising a D-galactose comprising oligosaccharide;
b. separating oligosaccharide from the legume material to provide an oligosaccharide composition, wherein the oligosaccharide composition comprises at least about 20 percent of the D-galactose comprising oligosaccharide relative to the legume material;
c. treating the oligosaccharide composition to purify the oligosaccharide;
d. hydrolyzing oligosaccharide to provide a D-galactose preparation.

2. The process according to claim 1, wherein the oligosaccharide composition is an aqueous solution and wherein treating the oligosaccharide composition comprises at least one of solvent-aided crystallization, nano-filtration and fermentation.

3. The process according to claim 2, comprising adding to the solution a water-soluble solvent, whereby oligosaccharides precipitate, and separating precipitated oligosaccharides from impurities remaining in solution.

4. The process according to claim 2, wherein treating the oligosaccharide composition comprises nano-filtration whereby oligosaccharides are retained on the membrane and impurities of lower molecular weight permeate.

5. The process according to claim 2, wherein treating the oligosaccharide composition comprises fermenting at least one of mono saccharides, disaccharides and other carbon molecules in the oligosaccharide composition.

6. The process according to claim 1, further comprising the step of treating the oligosaccharide composition by fermentation prior to the hydrolysis, after it or simultaneously with it.

7. The process according to claims 2, 5 or 6, comprising fermenting glucose, fructose and sucrose.

8. The process according to claims 2, 5, 6 or 7, wherein fermenting generates a fermentation product.

9. The process according to claim 8, wherein the fermentation product is selected from a group consisting of ethanol, carboxylic acids, amino acids, single-cell protein, enzymes and combinations thereof.

10. The process according to claim 9, wherein the fermentation product is an enzyme and the enzyme is used in the hydrolysis step.

11. The process according to claim 8, wherein the fermentation product is separated from oligosaccharides or from D-galactose.

12. A process for the production of a tagatose preparation comprising:
(a) providing a legume extract comprising at least one D-galactose-comprising oligosaccharide;
(b) hydrolyzing at least a fraction of the D-galactose-comprising oligosaccharide to form a D-galactose-comprising aqueous solution;
(c) isomerizing at least a fraction of the D-galactose in the aqueous solution to form a tagatose-comprising aqueous solution; and
(d) purifying the tagatose in the solution.

13. The process according to claim 12, wherein the legume is selected from a group consisting of soy, rape, lupin, sunflower, cowpea, and combinations thereof.

14. The process according to claim 12, wherein the extract contains water-soluble components of the legume.

15. The process according to claim 12, wherein the extract is formed on extraction of solubles from defatted beans.

16. The process according to claim 12, wherein the extract is a byproduct of manufacturing purified soy proteins.

17. The process according to claim 12, wherein the extract is selected from a group consisting of soy solubles, soy whey, soy molasses and combinations thereof.

18. The process according to claim 12, further comprising at least one additional purification step.

19. The process according to claim 18, wherein the purification step separates impurities from at least one of D-galactose-comprising oligosaccharide, D-galactose and tagatose.

20. The process according to claim 12, further comprising a step of purifying the extract by removal of solutes other than D-galactose-containing oligosaccharide.

21. The process according to claim 12, further comprising a step of purifying the aqueous solution by removal of solutes other than D-galactose.

22. The process according to claims 12, 18, 19, 20 or 21, wherein the purification involves one or more means selected from a group consisting of crystallization of tagatose, crystallization of a tagatose complex, crystallization of non-tagatose carbohydrates, solvent-aided crystallization, chromatographic separation, ultra-filtration, nano-filtration, fermentation of non-tagatose sugars and combinations thereof.

23. The process according to claim 12, wherein at least about 60 percent of the oligosaccharides having the D-galactose moiety are converted to D-galactose in monosaccharide form.

24. The process according to claim 12, wherein at least about 70 percent of the oligosaccharides having the D-galactose moiety are converted to D-galactose in monosaccharide form.

25. The process according to claim 12, wherein at least about 80 percent of the oligosaccharides having the D-galactose moiety are converted to D-galactose in monosaccharide form.

26. The process according to claim 12, wherein at least about 30 percent by weight of the plurality of oligosaccharides in the extract have the D-galactose moiety.

27. The process according to claim 12, further comprising purifying the extract to at least about 30 percent D-galactose comprising oligosaccharides on a dry weight basis.

28. The process according to claim 12, further comprising purifying the extract to at least about 40 percent D-galactose comprising oligosaccharides on a dry weight basis.

29. The process according to claim 12, wherein at least 90 percent of the D-galactose comprising oligosaccharide contain no other sugar moiety but glucose or fructose.

30. A process for producing a D-galactose preparation and an isoflavones preparation, comprising:
a. providing an extract of defatted soy flakes;
b. hydrolyzing at least a fraction of D-galactose-containing oligosaccharide from the extract;
c. separating the extract into at least two streams, one of which is enriched in D-galactose compared with isoflavones and the other is enriched in isoflavones compared with D-galactose;
d. purifying the D-galactose-enriched stream;
e. purifying the isoflavones-enriched stream.

31. The process according to claim 30, wherein the hydrolyzing is conducted prior to the separating, after it or simultaneously with it.

32. A process for producing a tagatose preparation and an isoflavones preparation, comprising:
a. providing an extract of defatted soy flakes;
b. hydrolyzing at least a fraction of D-galactose-containing oligosaccharide from the extract;
c. isomerizing D-galactose to tagatose;
d. separating the extract into at least two streams, one of which is enriched in D-galactose or in tagatose compared with isoflavones and the other is enriched in isoflavones compared with D-galactose or tagatose;
e. purifying the D-galactose or tagatose-enriched stream;
f. purifying the isoflavones-enriched stream.

33. The process according to claim 32, wherein the hydrolyzing is conducted prior to the separating, after it or simultaneously with it.

34. The process according to claim 32, wherein the isomerizing is conducted prior to the separating, after it or simultaneously with it.

35. The process according to at least one of claims 30-34, wherein the separating involves one or more means selected from a group consisting of crystallization of tagatose or galactose, crystallization a tagatose complex, crystallization of non-tagatose carbohydrates and oligosaccharides, solvent-aided crystallization, chromatographic separation, ultra-filtration, nano-filtration, fermentation of non-tagatose sugars and combinations thereof.

36. A product selected from a group consisting of D-galactose preparation, D-tagatose preparation, isoflavones preparation and products thereof prepared according to any of the previous steps.

37. A product formed from a product of claim 36 by at least one treatment selected from a group consisting of purification, heating, hydrogenation, oxidation, esterification, etherification and combinations thereof.

38. A product according to claim 36 or 37 with substantially no lactose.

39. A product according to claim 36 or 37 substantially free of lactose.

40. A product according to claim 36 or 37 comprising at least about 100 ppm fructose.

41. A product according to claim 36 or 37 comprising at least about 10 ppm fructose.

42. Food products or cosmetic products comprising products according to at least one of claims 36-41.

43. A process of using products according to at least one of claims 36-41 in food preparation.
